(19) United States
(12) Patent Application Publication
(10) Pub. No.: US 2019/0343697 A1
(43) Pub. Date: Nov. 14, 2019

(54) DISPOSABLE LIMB COVER FOR USE IN A SHOWER

(71) Applicants: BRITTANY FARLEY, Puslinch (CA); Ed Lewis, Puslinch (CA)

(72) Inventors: BRITTANY FARLEY, Puslinch (CA); Ed Lewis, Puslinch (CA)

(21) Appl. No.: 15/973,608

(22) Filed: May 8, 2018

(51) Int. Cl. A61F 15/00; A41D 3/00; A41D 13/12

(52) U.S. Cl. CPC A61F 15/004; A41D 3/00; A41D 2400/52; A41D 13/1254; A41D 13/1245

(57) ABSTRACT

The disposable limb cover for use in a shower is a garment. The disposable limb cover for use in a shower is worn by a patient during a shower. The disposable limb cover for use in a shower is worn over an appendage of the patient. The disposable limb cover for use in a shower is a fluid impermeable structure that protects the appendage of the patient while showering. The disposable limb cover for use in a shower comprises an extremity sheath, a joint wrap, a sleeve, and a fastener. The joint wrap attaches the extremity sheath to the sleeve. The joint wrap and the fastener bind the disposable limb cover for use in a shower to the patient.

### CROSS REFERENCES TO RELATED APPLICATIONS

Not Applicable

### STATEMENT REGARDING FEDERALLY SPONSORED RESEARCH

Not Applicable

### REFERENCE TO APPENDIX

Not Applicable

### BACKGROUND OF THE INVENTION

### Field of the Invention

The present invention relates to the field of medical and veterinary science including bandages and dressings, more specifically, a water impermeable dressing configured for use with a limb.

### SUMMARY OF INVENTION

The disposable limb cover for use in a shower is a garment. The disposable limb cover for use in a shower is worn by a patient during a shower. The disposable limb cover for use in a shower is worn over an appendage of the patient. The disposable limb cover for use in a shower is a fluid impermeable structure that protects the appendage of the patient while showering. The disposable limb cover for use in a shower comprises an extremity sheath, a joint wrap, a sleeve, and a fastener. The joint wrap the fastener bind the disposable limb cover for use in a shower to the patient.

These together with additional objects, features and advantages of the disposable limb cover for use in a shower will be readily apparent to those of ordinary skill in the art upon reading the following detailed description of the presently preferred, but nonetheless illustrative, embodiments when taken in conjunction with the accompanying drawings.

In this respect, before explaining the current embodiments of the disposable limb cover for use in a shower in detail, it is to be understood that the disposable limb cover for use in a shower is not limited in its applications to the details of construction and arrangements of the components set forth in the following description or illustration. Those skilled in the art will appreciate that the concept of this disclosure may be readily utilized as a basis for the design of other structures, methods, and systems for carrying out the several purposes of the disposable limb cover for use in a shower.

It is therefore important that the claims be regarded as including such equivalent construction insofar as they do not depart from the spirit and scope of the disposable limb cover for use in a shower. It is also to be understood that the phraseology and terminology employed herein are for purposes of description and should not be regarded as limiting.

### BRIEF DESCRIPTION OF DRAWINGS

The accompanying drawings, which are included to provide a further understanding of the invention are incorporated in and constitute a part of this specification, illustrate an embodiment of the invention and together with the description serve to explain the principles of the invention. They are meant to be exemplary illustrations provided to enable persons skilled in the art to practice the disclosure and are not intended to limit the scope of the appended claims.

FIG. 1 is a top view of an embodiment of the disclosure.

FIG. 2 is a bottom view of an embodiment of the disclosure.

FIG. 3 is a front view of an embodiment of the disclosure.

FIG. 4 is an in-use view of an embodiment of the disclosure.

FIG. 5 is an in-use view of an alternate embodiment of the disclosure.

FIG. 6 is a top view of an alternate embodiment of the disclosure.

### DETAILED DESCRIPTION OF THE EMBODIMENT

The following detailed description is merely exemplary in nature and is not intended to limit the described embodiments of herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description.

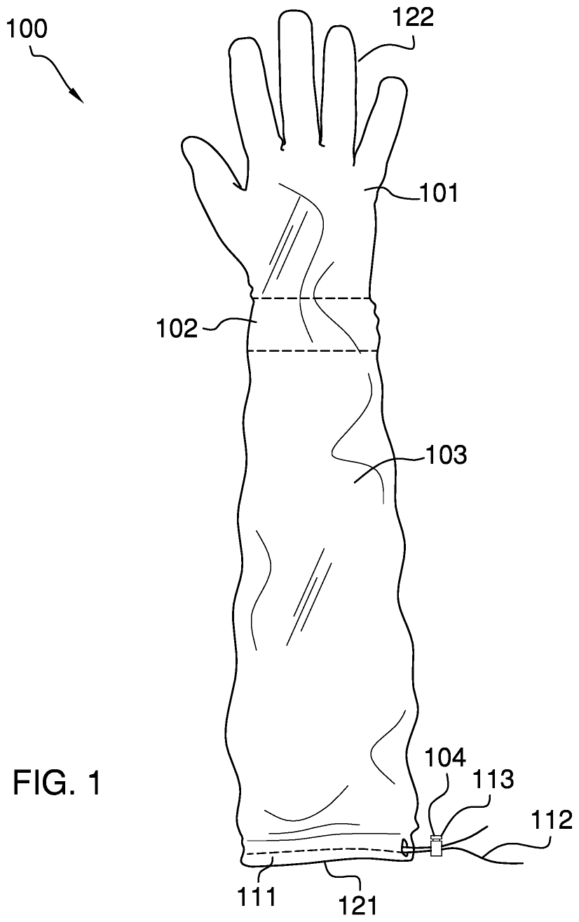

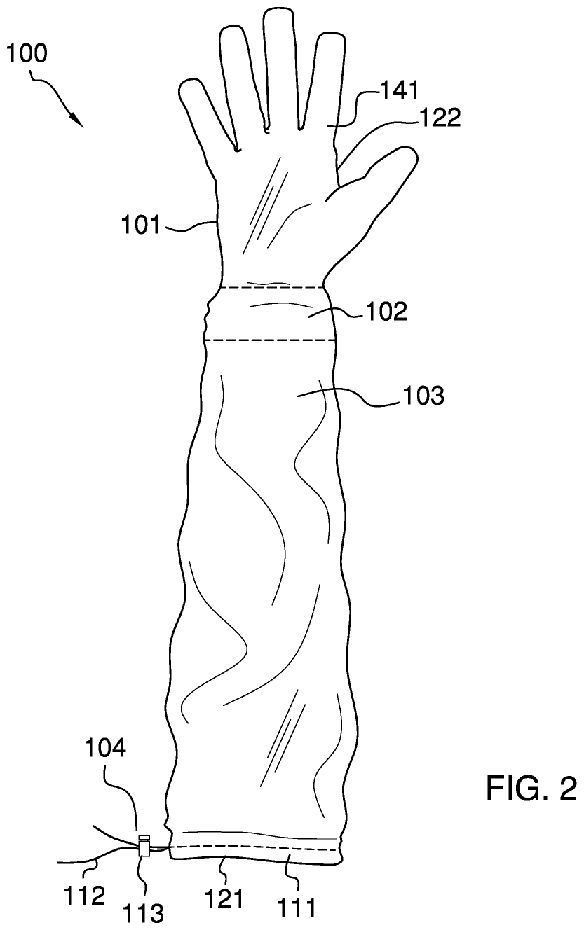

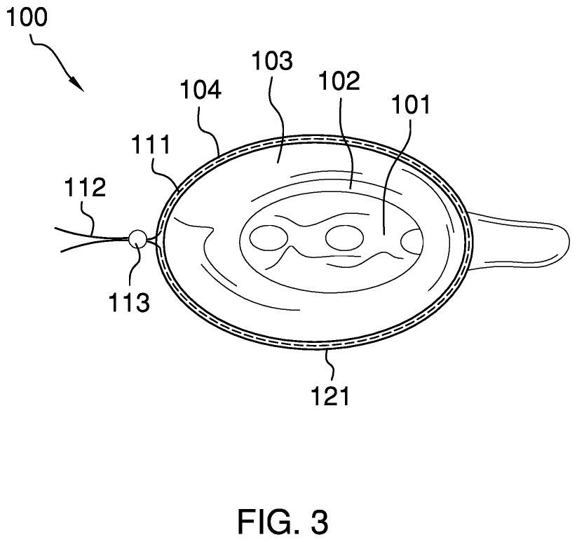

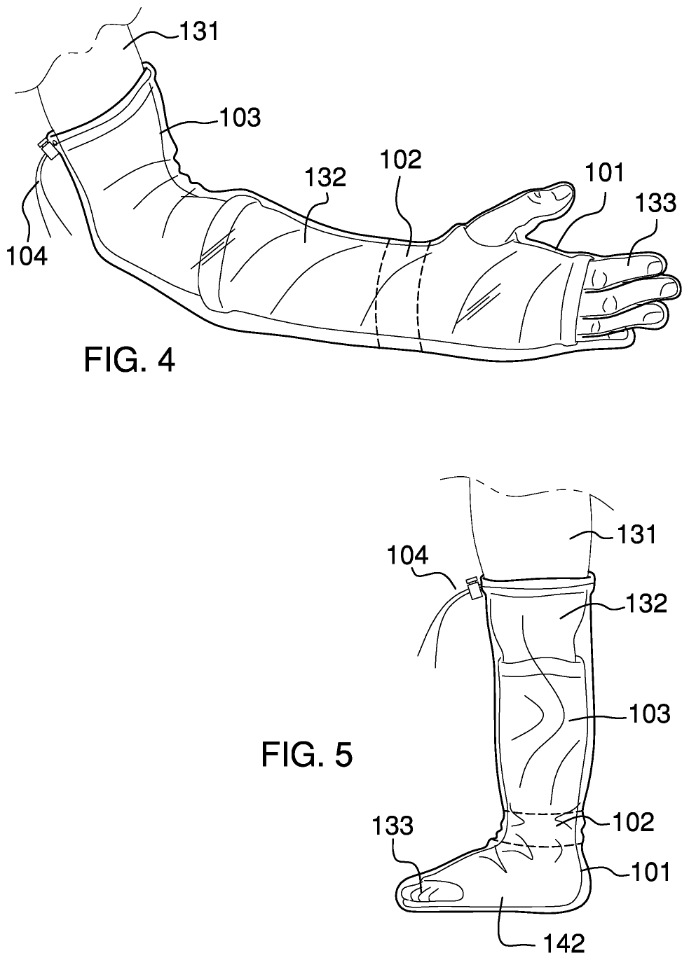

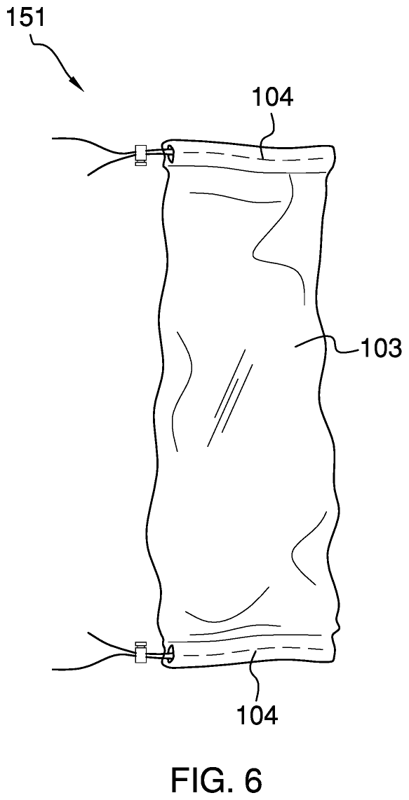

Detailed reference will now be made to one or more potential embodiments of the disclosure, which are illustrated in FIGS. 1 through 6.

The disposable limb cover for use in a shower 100 (hereinafter invention) is a garment. The invention 100 is worn by a patient 131 during a shower. The patient 131 is further defined with an appendage 132. The appendage 132 is further defined with an extremity 133. The extremity 133 is selected from the group consisting of a hand and a foot. The invention 100 is worn over an appendage 132 of the patient 131. The invention 100 is a fluid impermeable structure that protects the appendage 132 of the patient 131 while showering. The invention sleeve 103, and a fastener 104. The joint wrap 102 attaches the extremity 133 sheath 101 to the sleeve 103. The joint wrap 102 and the fastener 104 bind the invention 100 to the patient 131. The invention 100 is a disposable item intended for a single use. The invention 100 is further defined with an open end 121 and a sealed end 122.

The open end 121 is the end of the invention 100 the appendage 132 inserts into. The open end 121 is formed with an aperture. The sealed end 122 is the end of the invention 100 that is distal from the open end 121. The extremity 133 sheath 101 is at the sealed end 122.

The patient 131 refers to the person using the invention 100. The appendage 132 is defined in greater detail elsewhere in this disclosure. The extremity 133 is defined in greater detail elsewhere in this disclosure. The patient 131 is further defined with a wrist and an ankle.

The extremity 133 sheath 101 is the portion of the invention 100 that encloses the extremity 133 of the appendage 132 of a patient 131. The extremity 133 sheath 101 forms a fluid impermeable barrier around the extremity 133 of the protected appendage 132. The extremity 133 sheath 101 is formed from a fluid impermeable sheeting. The fluid impermeable sheeting is a polymer sheeting. The fluid impermeable sheeting The extremity 133 sheath 101 is formed to fit the shape of the protected extremity 133. The extremity 133 sheath 101 is a garment selected from the group consisting of a glove 141 and a sock 142. Specifically, when the extremity 133 protected by the extremity 133 sheath 101 is a hand, the extremity 133 has the shape of a glove 141. Similarly, when the extremity 133 protected by the extremity 133 sheath 101 is a foot, the extremity 133 has the shape of a sock 142. The glove 141 is defined in greater detail elsewhere in this disclosure. The sock 142 is defined in greater detail elsewhere in this disclosure.

The sleeve 103 is the portion of the invention 100 that encloses the appendage 132 of the patient 131 during use of the invention 100. The sleeve 103 forms a fluid impermeable barrier around the protected appendage 132 of the patient 131. The sleeve 103 is formed from a fluid impermeable sheeting. The fluid impermeable sheeting is a polymer sheeting. The fluid impermeable sheeting is an inelastic sheeting. The sleeve 103 has a tubular shape. The appendage 132 of the patient 131 inserts through the open end 121 of the invention 100 to don the invention 100.

The joint wrap 102 is a tubular section of the invention 100. The joint wrap 102 joins the extremity 133 sheath 101 to The joint wrap 102 forms a fluid impermeable barrier around the joint that attaches the extremity 133 to the appendage 132. The joint is selected from the group consisting of a wrist and an ankle. The joint wrap 102 is formed from a fluid impermeable sheeting. The fluid impermeable sheeting is a polymer sheeting. The fluid impermeable sheeting is an elastic sheeting. The joint wrap 102 is formed as an elastic tubular structure. In the first potential embodiment of the disclosure, a first fluid impermeable heat bond seam joins the joint wrap 102 to the extremity 133 sheath 101. A second fluid impermeable heat bond seam joins the joint wrap 102 to the sleeve 103.

The joint wrap 102 acts as a spring. Specifically, when a joint selected from the group consisting of a wrist and an ankle inserts into the joint wrap 102, a radial force is applied to the joint wrap 102 in a direction perpendicular to the center axis of the joint wrap 102. The applied radial force elongates the span of the diameter of the joint wrap 102 in the direction perpendicular to the center axis of the joint wrap 102. The elasticity of the joint wrap 102 creates a force that opposes the displacement created by the applied force. The elasticity of the joint wrap 102 returns the joint wrap 102 to its relaxed shape. The selected joint will prevent the joint wrap 102 from returning to its relaxed shape. In this circumstance, the joint center axis of the joint wrap 102 that binds joint wrap 102 to the selected joint. By binding the joint wrap 102 to the selected joint, the extremity 133 sheath 101 is held in a fixed position over the extremity 133 during use of the invention 100.

The fastener 104 is a fastening device that binds the open end 121 of the sleeve 103 to the appendage 132 of the patient 131. The fastener 104 comprises a rouleau 111, an elastic cord 112, and a cord lock 113.

The rouleau 111 is a channel that is formed around the perimeter of the open end 121 of the sleeve 103. The rouleau 111 is defined in greater detail elsewhere in this disclosure.

The cord lock 113 is a commercially available device that secures the two ends of the elastic cord 112 into a fixed position when the elastic cord 112 is placed under tension around the extremity 133. The cord lock 113 is defined in greater detail elsewhere in this disclosure.

The elastic cord 112 is a commercially available cord with formed one or more elastic yarns. The elastic cord 112 wraps around the appendage 132 to bind the open end 121 of the sleeve 103 to the appendage 132. The elastic cord 112 is defined in greater detail elsewhere in this disclosure.

The elastic cord 112 acts as a spring. Specifically, when a force is applied to both ends of the elastic cord 112 in a the applied force elongates the span of the end to end length the elastic cord 112 in the direction parallel to the center axis of the elastic cord 112. The elasticity of the elastic cord 112 creates a force that opposes the displacement created by the applied force. The elasticity of the elastic cord 112 returns the elastic cord 112 to its relaxed shape. When the elongated elastic cord 112 loops around an appendage 132 of the patient 131, the appendage 132 will prevent the elastic cord 112 from returning to its relaxed shape. In this circumstance, the elastic cord 112 will apply a force projecting radially away from the center axis of the elastic cord 112 and through the lateral face of the elastic cord 112 and against the appendage 132. This force binds the elastic cord 112 to the appendage 132 of the patient 131.

The use of a rouleau 111, an elastic cord 112, and a cord lock 113 is well-known and documented in the apparel and textile arts.

In a second potential embodiment of the disclosure, as shown most clearly in FIG. 6, the invention 100 further comprises an extension 151. The extension 151 comprises a second instantiation of the sleeve 103 and two instantiations of the fastener 104. The extension 151 extends the coverage of the the initial instantiation of the invention 100 is insufficient for this purpose.

The following definitions were used in this disclosure:

And/Or: As used in this disclosure, the term and/or is a grammatical conjunction that implies an inclusive or. Specifically, the term and/or implies that at least one and potentially more than one of the plurality of statements joined by the and/or conjunction will be true.

Appendage: As used in this disclosure, an appendage is a generic term used to describe either the arm and/or leg of a person.

Bind: As used in this disclosure, to bind is a verb that means to tie or secure a first object to a second object using a cord or webbing.

Capped Tube: As used in this disclosure, a capped tube is a tube with one closed end and one open end.

Center: As used in this disclosure, a center is a point that is: 1) the point within a circle that is equidistant from all the points of the circumference; 2) the point within a regular polygon that is equidistant from all the vertices of the regular polygon; 3) the point on a line that is equidistant from the ends of the line; 4) the point, pivot, or axis around which something revolves; or, 5) the centroid or first moment of an area or structure. In cases where the appropriate definition or definitions are not obvious, the fifth option should be used in interpreting the specification.

Center Axis: As used in this disclosure, the center axis is the axis of a cylinder or a prism. The center axis of a prism is the line that joins the center point of the first congruent face of the prism to the center point of the second corresponding congruent face of the prism. The center axis of a pyramid refers to a line formed through the apex of the pyramid that is perpendicular to the base of the pyramid. When the center axes of two cylinder, prism or pyramidal structures share the same line they are said to be aligned. When the center axes of two cylinder, prism or pyramidal structures do not share the same line they are said to be offset.

Cord: As used in this disclosure, a cord is a long, thin, flexible, and prism-shaped string, line, rope, or wire. Cords are made from yarns, piles, or strands of material that are braided or twisted together or from a monofilament (such as fishing line). Cords have tensile strength but are too flexible to provide compressive strength and are not suitable for use in pushing objects. String, line, cable, and rope are synonyms for cord.

Cord Lock: As used in this disclosure, a cord lock is a device that is used to tighten cords or drawstrings without the use of knots.

Disposable: As used in this disclosure, disposable is an adjective that refers to an object that is designed and intended for a single use. Within this context, an object would be considered disposable if it is not reusable after its initial use.

Don: As used in this disclosure, to don means to enclose a person or object within a structure. For example, a person dons a helmet.

Elastic: As used in this disclosure, an elastic is a material or object that deforms when a force is applied to it and that is able to return to its relaxed shape after the force is removed. A material that exhibits these qualities is also referred to as an elastomeric material.

Elastic Cord: As used in this disclosure, an elastic cord is a cord that contains elastic yarns as some of the yarns that make up the cord. An elastic cord is constructed such that the elastic cord will stretch when a force is applied and will return to its original shape when after the force is removed. Shock cord and bungee cord are synonyms for elastic cord.

Elastic Yarn: As used in this disclosure, an elastic yarn is a yarn formed from elastomeric materials.

Extremity: As used in this disclosure, extremity is a generic term used to describe either the hand and/or foot of a person.

Fastener: As used in this disclosure, a fastener is a device that is used to join or affix a first object to a second object.

Glove: As used in this disclosure, a glove is an item of apparel that covers a hand. The glove comprises five finger stalls into which the fingers of the hand insert. A glove is further defined with a palm side and a back side. The palm side is proximal to the palm of the hand. The back side is distal from the palm side.

Instantiation: As used in this disclosure, an instantiation refers to a specific physical object that is created using a specification.

Loop: As used in this disclosure, a loop is the length of a first structure including, that is folded or curved to form a closed or nearly closed space such that a linear structure such as a second line, a cord or a hook can be inserted through the space formed within the first structure. Within this disclosure, the first structure is said to be looped around the linear structure.

Patient: As used in this disclosure, a patient is a person who is designated to receive a medical treatment, therapy or service. The term patient may be extended to an animal when used within the context of the animal receiving veterinary treatment or services

Prism: As used in this disclosure, a prism is a three-dimensional geometric structure wherein: 1) the form factor of two faces of the prism are congruent; and, 2) the two congruent faces are parallel to each other. The two congruent faces are also commonly referred to as the ends of the prism. The surfaces that connect the two congruent faces are called the lateral faces. In this disclosure, when further description is required a prism will be named for the geometric or descriptive name of the form factor of the two congruent faces. If the form factor of the two corresponding faces has no clearly established or well-known geometric or descriptive name, the term irregular prism will be used. The center axis of a prism is defined as a line that joins the center point of the first congruent face of the prism to the center point of the second corresponding congruent face of the prism. The center axis of a prism is otherwise analogous to the center axis of a cylinder. A prism wherein the ends are circles is commonly referred to as a cylinder.

Relaxed Shape: As used in this disclosure, a structure is considered to be in its relaxed state when no shear, strain, or torsional forces are being applied to the structure.

Rouleau: As used in this disclosure, a rouleau is a tube or channel formed on the edge of a textile or sheeting.

Seam: As used in this disclosure, a seam is a joining of: 1) a first textile to a second textile; 2) a first sheeting to a second sheeting; or, 3) a first textile to a first sheeting. Potential methods to form seams include, but are not limited to, a sewn seam, a heat bonded seam, an ultrasonically bonded seam, or a seam formed using an adhesive.

Sheath: As used in this disclosure, a sheath is a shell that is used to cover an object and from which the object may be inserted and withdrawn.

Sheeting: As used in this disclosure, a sheeting is a material, such as a textile, a plastic, or a metal foil, in the form of a thin flexible layer or layers.

Sleeve: As used in this disclosure, a sleeve is a tube-like covering that is placed over a rod, shaft or other cylindrical object.

Sock: As used in this disclosure, a sock is a tubular garment worn over the foot and leg of a person. The sock is formed in the shape of a capped tube.

Spring: As used in this disclosure, a spring is a device that is used to store mechanical energy. This mechanical energy will often be stored by: 1) deforming an elastomeric material that is used to make the device; 2) the application of a torque to a rigid structure; or 3) a combination of the previous two items.

Tube: As used in this disclosure, the term tube is used to describe a hollow prism structure with two open faces. In this disclosure, the terms inner dimension and outer dimension of a tube are used as they would be used by those skilled in the plumbing arts.

Yarn: As used in this disclosure, a yarn is a continuous strand of textile fibers and filaments. Yarns are generally used in the production of fabrics. For the purposes of this disclosure, this definition explicitly includes yarns formed from a single filament such as a monofilament yarn.

With respect to the above description, it is to be realized that the optimum dimensional relationship for the various components of the invention described above and in FIGS. 1 through 6 include variations in size, materials, shape, form, function, and manner of operation, assembly and use, are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

What is claimed is:

1. A fluid impermeable dressing comprising:
an extremity sheath, a joint wrap, a sleeve, and a fastener;
wherein the joint wrap attaches the extremity sheath to the sleeve;
wherein the joint wrap and the fastener bind the fluid impermeable dressing to the patient;
wherein the fluid impermeable dressing is a garment;
wherein the fluid impermeable dressing is worn by a patient;
wherein the patient is further defined with an appendage;
wherein the appendage is further defined with an extremity;
wherein the extremity is selected from the group consisting of a hand and a foot;
wherein the hand is further defined with a wrist;
wherein the foot is further defined with an ankle;
wherein the fluid impermeable dressing is worn over an appendage of the patient;
wherein the fluid impermeable dressing is a fluid impermeable structure that protects the appendage of the patient from water;
wherein the fluid impermeable dressing is a disposable item;
wherein the fluid impermeable dressing is further defined with an open end and a sealed end.

2. The fluid impermeable dressing according to claim 1
wherein the extremity sheath is the portion of the fluid impermeable dressing that encloses the extremity of the appendage of a patient;
wherein the extremity sheath forms a fluid impermeable barrier around the extremity of the protected appendage.

3. The fluid impermeable dressing according to claim 2
wherein the sleeve is the portion of the fluid impermeable dressing that encloses the appendage of the patient;
wherein the sleeve forms a fluid impermeable barrier around the protected appendage of the patient.

4. The fluid impermeable dressing according to claim 3
wherein the joint wrap joins the extremity sheath to the sleeve to form the sealed end of the fluid impermeable dressing;
wherein the joint wrap forms a fluid impermeable barrier around a joint that attaches the extremity to the appendage;
wherein the joint is selected from the group consisting of the wrist and the ankle.

5. The fluid impermeable dressing according to claim 4 wherein the fastener binds the sleeve to the appendage of the patient.

6. The fluid impermeable dressing according to claim 5
wherein the extremity sheath is formed from a fluid impermeable sheeting;
wherein the fluid impermeable sheeting is a polymer sheeting;
wherein the fluid impermeable sheeting is an inelastic sheeting.

7. The fluid impermeable dressing according to claim 6
wherein the extremity sheath is formed to fit the shape of the protected extremity;
wherein the extremity sheath is a garment selected from the group consisting of a glove and a sock;
wherein when the extremity protected by the extremity sheath is a hand then the extremity has the shape of a glove;
wherein when the extremity protected by the extremity sheath is a foot then the extremity has the shape of a sock.

8. The fluid impermeable dressing according to claim 7
wherein the sleeve is formed from a fluid impermeable sheeting;
wherein the fluid impermeable sheeting is a polymer sheeting;
wherein the fluid impermeable sheeting is an inelastic sheeting;

9. The fluid impermeable dressing according to claim 8 wherein the sleeve has a tubular shape.

10. The fluid impermeable dressing according to claim 9
wherein the joint wrap is formed from a fluid impermeable sheeting;
wherein the fluid impermeable sheeting is a polymer sheeting;
wherein the fluid impermeable sheeting is an elastic sheeting.

11. The fluid impermeable dressing according to claim 10 wherein the joint wrap is formed as an elastic tubular structure.

12. The fluid impermeable dressing according to claim 11
wherein a first fluid impermeable heat bond seam joins the joint wrap to the extremity sheath;
wherein a second fluid impermeable heat bond seam joins the joint wrap to the sleeve.

13. The fluid impermeable dressing according to claim 12
wherein the joint wrap is as a spring;
wherein the joint wrap will apply a force projecting radially towards the center axis of the joint wrap that binds joint wrap to the selected joint.

14. The fluid impermeable dressing according to claim 13 wherein joint wrap holds the extremity sheath in position over the extremity.

15. The fluid impermeable dressing according to claim 14
wherein the fastener comprises a rouleau, an elastic cord, and a cord lock;
wherein the elastic cord inserts into the rouleau;
wherein the cord lock attaches to the elastic cord.

16. The fluid impermeable dressing according to claim 15 wherein the rouleau is a channel formed around the perimeter of the open end of the sleeve.

17. The fluid impermeable dressing according to claim 16
wherein the elastic cord comprises one or more elastic yarns;
wherein the elastic cord is a spring;
wherein the elastic cord loops around the appendage;
wherein the elastic cord will apply a force projecting radially away from the center axis of the elastic cord and through the lateral face of the elastic cord and against the appendage to bind the elastic cord to the appendage.

18. The fluid impermeable dressing according to claim 17 wherein the cord lock secures the two ends of the elastic cord into a fixed position when the elastic cord is placed under tension.

19. The fluid impermeable dressing according to claim 18
wherein the fluid impermeable dressing further comprises an extension;
wherein the extension comprises a second instantiation of the sleeve and two instantiations of the fastener;
wherein the extension extends the coverage of the surface area protected by the primary instantiation fluid impermeable dressing.
